(19) United States
(12) Patent Application Publication
(10) Pub. No.: US 2005/0010086 A1
(43) Pub. Date: Jan. 13, 2005

(54) Process methods and apparatus for removing non-adherent elements for the skin of living beings, for measuring the hair loss of living beings, for measuring a quality sacle of a manufactured product and for having the pattern of non-adherent elements on a surface

(76) Inventor: Dominique Vanneste, Tournai (BE)

Correspondence Address: James C Wray, Suite 300, 1493 Chain Bridge Road, McLean, VA, 22101, US

(21) Appl. No.: 10/497,903

(22) PCT Filed: Jun. 12, 2002
(86) PCT No.: PCT/EP02/06434

(51) Int. Cl. A61B 17/50; A61B 5/00

(52) U.S. Cl. 600/300; 606/134

(57) ABSTRACT

Process, method and apparatus for removing non-adherent and/or loosely attached elements from the skin of living beings and measuring the hair loss of living beings. Process for removing non-adherent and/or loosely attached elements from the skin of a living being, comprising applying on said skin (1) an adhesive (10, 11) having an adhesive strength which is lower than the greatest anchorage strength of certain hairs (5) in said skin. Use of this process to measure hair loss from the skin of living beings or flux of hairs from the telogen phase to the exogen phase.

The present invention relates to a process for removing non-adherent or loosely attached elements from the skin of living beings.

The skin of living beings is frequently recovered with scales, excess of grease or similar particularly in very hairy regions of the body such as the scalp in case of human beings. Generally such scales are continuously formed on the skin during the whole life of the living being. It is further known that living beings continuously leave hairs during their life. Of course this loss of hairs is normally important in very hairy regions such as the scalp of human beings.

For hygienic and healthy reasons it is generally requested to remove periodically such non-adherent elements from the skin of living beings particularly human beings.

For skin and hair evaluation there is also a need for a method adapted to measure the hair loss of living beings, particularly of human beings. Indeed, hair loss (i.e. the amount of hair lost in one definite unit of time, for example one day, one week or one month) may be an information on the health of a human being or of an animal or on the influence of different factors on the health, such as standard of living.

It is known to use strips having an adhesive wax or similar thereon to remove unwanted hairs (U.S. Pat. No. 1 620 539). This known method is easy, rapid and inexpensive. However when using such strips all the hairs which are put in contact with the adhesive wax of the strip are removed from the skin. This may be a disadvantage for certain applications, particularly to remove scales or shedding hair from the scalp without damage to the hair fiber itself or the hair roots which are in a growing phase.

For certain applications, particularly for very hairy regions such as animal fleece or man and woman scalp there is thus a need for a means which is adapted to remove non-adherent elements from the skin (such as scales or shedding hairs) without damage for the hair.

Accordingly it is an aim of this invention to provide a process which is adapted to remove non-adherent elements from the skin of living beings without altering hairs. Another aim of this invention is to provide a process which is adapted to detach and remove hairs which are loosely attached to the skin of a living being.

A further aim of this invention is to provide a method to measure the hair loss from the skin of living beings.

An additional aim of this invention consists in providing methods and apparatus for skin and hair evaluation of living beings, particularly of human beings.

Accordingly the invention relates to a process for removing non-adherent and/or loosely attached elements from the skin of a living being, comprising applying an adhesive on said skin and tearing said adhesive from said skin, this process being characterized in that said adhesive is selected among those having an adhesive strength which is lower than the greatest anchorage strength of hairs in said skin.

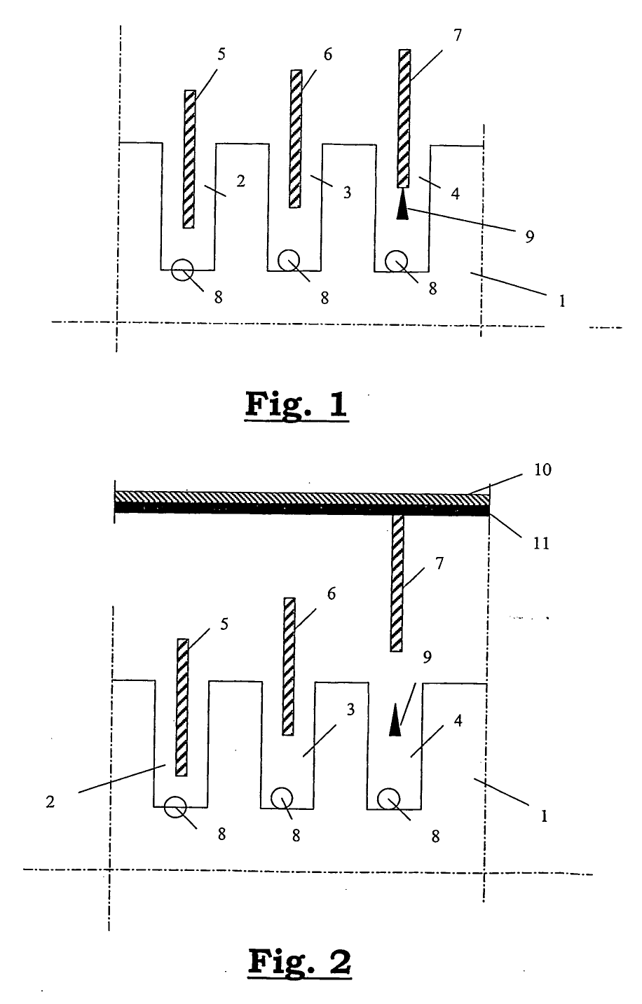

It is known that hairs grow from follicles that undergo cyclical phases of growth and rest. It is generally considered that the hair cycle comprises four successive phases. A first phase (the anagen phase) corresponds to an intense activity of the follicles with hair production and growth at the skin surface. The duration of the anagen phase varies greatly with the species and the age and from region to region of the body. The end of the anagen phase corresponds to an arrest in hair production and a shrinkage of the dermal papilla. It is followed by the so-called catagen phase where the dermal papilla undergoes an ascending movement together with the hair shaft. The catagen phase is thus characterized by an apparent elongation of the hair fiber which reflects the outward migration of the hair shaft. The catagen phase is completed when the dermal papilla abuts the bottom of the permanent portion of the hair follicle and no hair elongation is observed at the skin surface. It is followed by the telogen phase which is a true resting stage. During this third phase the dermal papilla expands and attracts epithelial cells from the bulge of the follicle in a downward movement. The epithelial cells then start production of a new non-pigmented hair fiber. Under physiological conditions this may correspond morphologically in timing to the end of the telogen phase and the beginning of the exogen phase of the previous hair cycle terminating with hair shedding. [Handbook of Cosmetic Science and Technology; A. O. Barel, M. Paye and H. I. Maibach editors, Marcel Dekker, Inc. New York Basel, 2001, pages 35-46: Ghassan Shaker and Dominique Van Neste (Skinterface sprl, Tournai, Belgium), “Hair” (FIG. 1 of pages 38 and 39)]. The anchorage strength of an hair fiber in the skin varies with time. In case of human beings it generally decreases from the anagen to the exogen phase: during the anagen and the catagen phases the hairs are generally strongly anchored in the skin; the anchorage of the hairs in the skin is less strong during the telogen phase and it is very week during the exogen phase. However this is not a generality for all body sites of all living beings. For some animals (notably telogen furs) or some body sites of human beings (such as eyebrows or eyelashes) the anchorage strength of hairs in the telogen phase may be greater than the anchorage strength of the hairs which are in the anagen phase.

The duration of the hair cycle is influenced by a series of factors such as species, age, standard of living and region of the skin (Rodney Dawber & Dominique Van Neste, “Hair and Scalp Disorders” Martin Dunitz Editors, London, 1995 pages 11-14; Dominique Van Neste & Hugh Rushton “Hair Problems in Women”; Elsevier Science Inc.; 1997; pages 113-114).

In this invention, “non-adherent elements” are elements which do not belong or will not interact for long with the surrounding living skin and are at most loosely attached to the skin. They may comprise dust, solidified particles of sebum or of coagulated blood, detached hairs and any similar elements.

“Non-adherent” means that these elements are either not attached to the skin or only loosely attached to the skin. “Loosely attached elements” comprise non adherent elements as just defined hereabove as well as elements which are at least partly inside a follicular orifice and at most loosely attached to the skin inside said follicular orifice. Such elements may comprise hairs or any other fibers for example artificial hair.

“Loosely attached to the skin” means that the attachment strength of these elements to the skin is lower than the greatest anchorage strength of living hair follicles that produce hairs in said skin. So in case of human beings, the attachment strength of these elements to the skin is lower than the anchorage strength of the hairs which are in the anagen phase.

In the process according to the invention the adhesive is any means adapted to seize or catch said non-adhesive elements. It may be glue. However preferred adhesives comprise elastic macromolecular matrices. With an elastic macromolecular matrix the non-adhesive elements and the hairs are seized and anchored in the matrix and there is competitiveness between the anchorage strength of the hair fibers in the follicles and the elasticity of the polymeric matrix and this competitiveness allows a selective removal in function of the anchorage strength. Polymers with decreasing elasticity allow selective removal in function of the anchorage of the fiber which will be removed. Generally silicon-based adhesives are convenient.

In an advantageous embodiment of the process according to the invention the adhesive is used as a coating on a strip, preferably a flexible strip, for example a tissue or a plastic sheet.

The process according to the invention is particularly adapted to remove hairs which are in the aforesaid exogen phase of the hair cycle.

Thus according to a particular embodiment of this invention in case of human beings the adhesive is selected among those having an adhesive strength which is higher than the anchorage strength of the hairs in exogen phase but lower than the anchorage strength of the hairs which are in the telogen phase. This embodiment of the invention is adapted to remove the hairs which are in exogen phase while healthy hairs which are in the anagen, catagen and telogen phases are substantially not affected.

This particular embodiment of the invention is particularly adapted to remove hairs which, although being in the exogen phase, are nevertheless temporarily retained inside the follicular orifices by a gluing strength (the so-called trichostasis). In this particular embodiment of the invention, such exogen hairs retained by trichostasis in follicular orifices are aforesaid “loosely attached elements”.

This particular embodiment of this invention is further adapted to capture disordered anagen follicles as a result of inflammation or any physical and/or chemical damage (for example laser treatment, radiotherapy, chemotherapy, etc.). The origin of the agents and/or phenomenon which are responsible of these disordered follicles may be either internal or external to the body of the living being.

A second aim of the invention consists in the use of the invention process defined above for pealing away the skin of living beings (particularly human beings) or for thinning hairs of human beings or animals. The invention process defined above is indeed adapted to clean skin from any unwanted elements such as scales, dusts and solidified particles of sebum or blood. It may be used for thinning the hair of men or women by removing hairs from the scalp which are in an exogen phase as well as damaged anagen and/or catagen hairs if any without damage to healthy hairs which are in either the anagen, the catagen or the telogen phases.

The process according to the invention has also an application in the preparation of a skin surface before making very fine photography thereof or for scientific studies of the hair cycles.

A third aim of the invention is the use of the invention process defined above to measure the hair loss of living beings, this use comprising counting (by any means known per se) the number of hairs which have been detached from the skin by said adhesive.

It is indeed known that hair loss is influenced by different factors, particularly the origin or species, age, sex, living conditions and standard of living, disease, medical treatment, physical treatment, chemical treatment, etc. The process according to the invention is well adapted to measure hair loss of living beings and so to appreciate the influence of each or a plurality of the aforesaid factors on the hair cycle and the loss of hair.

Accordingly a further object of the invention is a method to measure the hair loss from the skin of living beings, this method comprising in a first step applying on said skin an adhesive selected among those having an adhesive strength which is higher than the anchorage strength of hairs in exogen phase but lower than the anchorage strength of the least anchored hairs other than those which are in exogen phase; in a second step tearing said adhesive from said skin and in a third step counting the amount of hairs which have been detached from the skin in said second step.

In the measuring method according to the invention, the adhesive may advantageously be a calibrated coating on a strip.

In a preferred embodiment of the measuring method according to the invention, this measuring method includes at least three successive phases, comprising
(a) a first phase which comprises the aforesaid first, second and third steps; and
(b) at least one second phase which comprises repeating the aforesaid first, second and third steps of the first phase (a) with another adhesive having substantially the same adhesive strength as the one of the adhesive of the first phase (a); and
(c) a final phase which comprises doing the sum of the amounts of hairs which have been counted respectively in said first phase (a) and in said second phase (b).

An advantageous modification of this preferred embodiment includes
(d) after said second phase (b) and before said final phase (c), at least one additional phase which comprises repeating the aforesaid first, second and third steps of the first phase (a) with an additional adhesive having substantially the same adhesive strength as the one of the adhesives of the first phase (a) and the second phase (b); and
(e) the aforesaid final phase (c) comprises doing the sum of the amounts of hairs which have been counted respectively in said first phase (a), in said second phase (b) and in said at least one additional phase (d).

In this advantageous modification just described, said at least one additional phase (d) may either consist in a single third phase or include a third and a fourth (and possibly a fifth and even more than a fifth) phase.

The measuring method according to the invention has several applications for animals and human beings.

When the measuring method according to the invention is used for measuring hair loss of human beings said least anchored hairs are the hairs which are in the telogen/exogen physiological phase. Accurate analysis of hair loss of animals or human beings is more and more requested to have a better knowledge of the action of different parameters or factors on the loss of hairs, such as the species, age, sex and living conditions. Thus in addition to the analysis of the hair loss from the scalp of living beings (particularly human beings) the measuring method according to the invention allows to measure parameters of the detached hairs such as for example the thickness, length, color, genetic material, etc.

In this application of the measuring method of the invention it may be interesting to obtain a pattern of hair loss from the skin of living beings, particularly from the hair scalp of human beings. It is indeed known that the loss of hairs from the scalp is not uniform on the surface of the scalp but varies greatly from region to region of the scalp. There is thus a need for a knowledge of the topographical distribution of the hairs on men or women scalp, and particularly for the modifications in this topographical distribution with time or other physical or physiological factors.

Thus, in an improved embodiment of the measuring method according to the invention, this method comprises dividing the skin in a plurality of zones before said first step, and said first, second and third steps are executed successively on each of said plurality of zones.

In this improved embodiment of the method according to the invention, a specific means to divide the skin in a plurality of zones may be to dispose a mask on the skin, this mask having openings separated by strips of a material which is impervious to the adhesive applied in the first step of the method. So the adhesive used in said first step will pass only through the openings of the mask and attain the scalp.

In this aforesaid improved embodiment of the measuring method according to the invention, it is preferred to spread a marking. This marking product may advantageously be a liquid dye. In this preferred embodiment of the invention the dye is selected among those having an adhesive strength or surface tension which is lower than the detachment strength of the adhesive in the second step of the method. So the marking product will attain and remain only in the follicular orifices of the skin where hairs have been removed from in the second step (or steps) of the method. This will allow a better visual appearance of hair loss on the skin. Any liquid dye having no detrimental action on the hairs and the skin may be used. Examples of liquid dyes comprise eosin, fluorescein, paraphenylene derivatives, etc. (non-exhaustive list).

The invention concerns also an apparatus to achieve the aforesaid improved embodiment of the measuring method according to the invention, said apparatus comprising a mask, means to apply said mask on a specific zone of the skin, means to apply an adhesive on said specific zone of the skin in said mask, means to tear said adhesive from the skin and means to count hairs detached by the adhesive.

In the apparatus according to the invention the adhesive may be a liquid one. In this embodiment of the apparatus of the invention, the means to apply the adhesive on the skin may be a brush or a spraying device or a spatula or similar.

In another embodiment of the apparatus according to this invention the means to apply the adhesive on the skin comprises a flexible strip having a coating of said adhesive thereon.

In the apparatus according to the invention any convenient means may be used to count hairs detached by the adhesive.

The apparatus according to the invention is adapted to obtain the pattern of hair loss of animals or human beings. It is particularly well adapted to have a pattern of scalp hair loss of men or women.

The measuring method and apparatus according to the invention may be used with advantage to measure the flux of hairs passing from the telogen to the exogen phase, i.e. the amount of hairs which, on a definite surface area of the skin (for example a definite area of the head of a human being), pass from the exogen phase to the telogen phase in a definite unit of time (for example in one hour, ten hours, one day, two (or more) days, one week or more, one month or more, etc.).

The invention concerns thus also a method to measure the flux of hairs from the telogen phase to the exogen phase on a definite surface area of the skin of a living being, said method comprising
(a) at least a first series of steps which includes
   a first step which comprises applying on said surface area an adhesive selected among those having an adhesive strength which is higher than the anchorage strength of hairs in exogen phase but lower than the anchorage strength of the least anchored hairs other than those which are in exogen phase; and
   a second step which comprises tearing said adhesive from said surface area; and
(b) a second series of steps which follows said at least first series of steps (a) and which includes
   a first step which comprises repeating said first and second steps of said at least first series of steps (a) with another adhesive having an adhesive strength substantially equal to the one of the adhesive of said at least first series of steps (a);
   a second step which comprises counting the amount of hairs which have been detached from the skin surface area by said another adhesive; and
   a third step which comprises dividing said amount of hairs by the surface area and by the time elapsed between said tearing steps of both the first (a) and the second (b) series of steps.

In case of human beings, said time in said third step of said second series of steps (b) may advantageously be of from at least one day to at most one month.

The characteristics of the invention disclosed above can be adapted, without any inventive modification, to the measure of the quality of any heterogeneous surface comprising fibers of which some could be or become loosely attached to a support.

Accordingly the applicant considers that the use of the characteristics of its measuring method and apparatus to such a measure is inside the scope of this patent application.

For a more complete understanding of the invention and advantages thereof, reference is now made to the following description of preferred embodiments with reference to the attached drawings.

FIG. 1 is a schematic view of a vertical section through a portion of the scalp of a human being;

FIG. 2 shows the scalp of FIG. 1, using the process of this invention.

In these figures, same reference numerals designate same components.

In FIG. 1 a scalp portion 1 comprises follicles 2, 3 and 4 with hair fibers 5, 6 and 7. Reference numeral 8 designates dermal papilla. Hair fiber 5 is in an anagen phase and hair fiber 6 is in a telogen phase. In follicle 4 a new non-pigmented hair 9 is developing and hair fiber 7 is in an exogen phase.

In FIG. 2 a strip 10 comprising an adhesive 11 has first been applied on the scalp portion 1 and thereafter tore away therefrom. According to the invention the adhesive 11 is an elastic polymeric matrix having an adhesive strength to the hairs which is higher than the anchorage strength of hair fiber 7 in its follicle 4 but lower than the anchorage strength of the hair fibers 5 and 6 in their respective follicles 2 and 3. So after tearing of the strip 10 from the scalp 1 the hair fiber 7 (in exogen phase) is removed from the scalp and retained on the strip 10, while hair fibers 5 and 6 (respectively in anagen and telogen phases) remain anchored in the scalp 1.

1-26. (cancelled)

27. Process for removing non-adherent and/or loosely attached elements from the skin of a living being, comprising applying an adhesive on said skin and tearing said adhesive from said skin, wherein said adhesive is selected among those having an adhesive strength which is lower than the greatest anchorage strength of hairs in said skin.

28. Process according to claim 27, wherein said living being is a human being.

29. Process according to claim 28, wherein said greatest anchorage strength is the anchorage strength of hairs in anagen phase.

30. Process according to claim 27, wherein said adhesive strength of the adhesive is higher than the anchorage strength of hairs in exogen phase.

31. Process according to claim 30, wherein in case of a human being, said adhesive strength is lower than the anchorage strength of hairs in telogen phase.

32. Process according to claim 27, wherein said adhesive comprises an elastic macromolecular matrix.

33. Process according to claim 32, wherein said adhesive comprises a silicon-based macromolecular matrix.

34. Use of a process in accordance with claim 27, for peeling away said skin.

35. Use of a process in accordance with claim 27, for thinning hairs.

36. Method to measure the hair loss from the skin of living beings, comprising in a first step applying on said skin an adhesive selected among those having an adhesive strength which is higher than the anchorage strength of hairs in exogen phase but lower than the anchorage strength of the least anchored hairs other than those which are in exogen phase; in a second step tearing said adhesive from said skin and in a third step counting the amount of hairs which have been detached from the skin in said second step.

37. Method according to claim 36, wherein said counting the amount of hairs in said third step comprises counting the amount of hair on said adhesive after tearing thereof.

38. Method according to claim 36, wherein it comprises dividing the skin in a plurality of zones before said first step, and in that said first, second and third steps are executed successively on each of said plurality of zones.

39. Method according to claim 38, wherein it comprises applying a mask on every zones of said plurality of zones except the one on which said first, second and third steps are executed.

40. Method according to claim 38, wherein it further comprises spreading a marking product on said zones of the skin.

41. Method according to claim 40, wherein said marking product comprises a liquid dye.

42. Method according to any one 36, wherein said living beings are human beings.

43. Method according to claim 42, wherein said least anchored hairs are hairs in telogen phase.

44. Apparatus to achieve the method according to claim 39, comprising a mask, means to apply said mask on a specific zone of the skin, means to apply an adhesive on said specific zone of the skin in said mask, means to tear said adhesive from the skin and means to count hairs detached by the adhesive.

45. Apparatus according to claim 44, wherein said means to apply the adhesive on the skin comprises a flexible strip having a coating of said adhesive thereon.

46. Method to measure the flux of hairs from the telogen phase to the exogen phase on a definite surface area of the skin of a living being, comprising
(a) at least a first series of steps which includes:
a first step which comprises applying on said surface area an adhesive selected among those having an adhesive strength which is higher than the anchorage strength of hairs in exogen phase but lower than the anchorage strength of the least anchored hairs other than those which are in exogen phase; and
a second step which comprises tearing said adhesive from said surface area; and
(b) a second series of steps which follows said at least first series of steps (a) and which includes:
a first step which comprises repeating said first and second steps of said at least first series of steps (a) with another adhesive having an adhesive strength substantially equal to the one of the adhesive of said at least first series of steps (a);
a second step which comprises counting the amount of hairs which have been detached from the skin surface area by said another adhesive; and
a third step which comprises dividing said amount of hairs by the surface area and by the time elapsed between said tearing steps of both the first (a) and the second (b) series of steps.

47. Method according to claim 46, wherein said time is from at least one day to at most one month.
